(19) United States
(12) Patent Application Publication
(10) Pub. No.: US 2019/0021651 A1
(43) Pub. Date: Jan. 24, 2019

(54) SYSTEM FOR INTRAORAL MEASUREMENT OF JAW DISPLACEMENTS

(71) Applicant: HICAT GMBH, BONN (DE)

(72) Inventors: NILS HANSSEN, BONN (DE); JOACHIM HEY, KOENIGSWINTER (DE); JOCHEN KUSCH, WACHTBERG (DE)

(73) Assignee: HICAT GMBH, BONN (DE)

(21) Appl. No.: 16/078,177

(22) PCT Filed: Feb. 23, 2017
(86) PCT No.: PCT/EP2017/054162

§ 371 Date: Aug. 21, 2018

(30) Foreign Application Priority Data
Feb. 25, 2016 (DE) 10 2016 103 320.0

(51) Int. Cl. A61B 5/00; A61B 5/11

(52) U.S. Cl. CPC A61B 5/4542; A61B 5/0826; A61B 5/0077; A61B 5/1127

(57) ABSTRACT

A method for capturing patient-individual movements of a mandible in relation to a maxilla in a plurality of degrees of freedom. The method includes providing an optical sensor system having an image in an oral cavity of a patient, providing a defined object in the image of the optical sensor system in the oral cavity of the patient, bringing the optical sensor system into a fixed connection to the mandible or to the maxilla, capturing the defined object with the optical sensor system, the defined object having a defined relationship to the other maxilla or mandible, recording a sequence of spatial points of the defined object with the optical sensor system during a movement of the mandible, and storing the sequence of spatial points as a multi-dimensional movement line in a movement data record.

### CROSS REFERENCE TO PRIOR APPLICATIONS

This application is a U.S. National Phase application under 35 U.S.C. § 371 of International Application No. PCT/EP2017/054162, filed on Feb. 23, 2017 and which claims benefit to German Patent Application No. 10 2016 103 320.0, filed on Feb. 25, 2016. The International Application was published in German on Aug. 31, 2017 as WO 2017/144585 A1 under PCT Article 21(2).

### FIELD

The present invention relates to a method and to a system for capturing patient-individual movements of the mandible in relation to the maxilla in a plurality of degrees of freedom.

### BACKGROUND

The displacement of the mandible in relation to the maxilla is of interest in various diagnostic situations and treatment plans. In the treatment of obstructive sleep apnea (OSA), for example, the mandible of the patient is displaced anteriorly by what is called a protrusion splint in order to widen the airways during sleep. The degree of anterior displacement is a compromise between the greatest possible widening of the airways and at the same time the least possible loading of the temporomandibular joints and of the surrounding soft tissue. In order to plan the protrusion splint virtually on a computer, it is essential to measure the patient-individual mandibular protrusion, since the protrusion in each patient is associated individually with a slight opening of the jaw and lateral movements. Knowledge of the individual protrusion patterns thus permits a therapy which is as effective as possible and which is gentle for the patient.

The degree of mandibular protrusion is conventionally measured with what is called a George gauge. This purely mechanical apparatus can only measure the overall extent of the anterior displacement; the recording of jaw opening movements and lateral movements along the protrusion distance remains unknown.

From the functional equipment available in treatment, measuring devices have previously been described with which individual mastication patterns can be recorded and, therefore, disturbances in the mastication function can be identified. These measurement apparatuses are also called condylographs, from the word condyle meaning joint. Condylographs can also be used to record protrusion movements of the jaw. They are, however, very complex to operate and, because of their size, are awkward to handle. A method called jaw motion tracking (JMT) makes use of a system consisting of a face bow with integrated receiver modules and a counterbalanced mandibular sensor which is fixedly connected to the mandibular teeth via a magnetic retainer on a para-occlusal retainer. This system records movement data, which are then superposed by three-dimensional graphics data of the mastication system that have been previously determined by an X-ray scan.

An intraoral system has previously been described in which the relative movements of the jaws are recorded by a measurement sensor which is held on the maxilla and which interacts with an electronic support pin register mounted in the mandible. The data are routed to the outside by a cable and are processed by an external computer. The power of the mastication muscles can then be determined from the measured mastication force.

A metric relationship to the geometry of the teeth cannot be produced, however, in this very straightforward system. On account of the cable routed to the outside, the patient cannot attain terminal occlusion and thus perform the protrusion. No aligners can additionally be measured. With this system, it is also not possible to measure all six degrees of freedom of the movement.

A disadvantage of some of the known systems is that a device connected rigidly to the teeth must be routed to the outside while being intended not to impede occlusion. This is in most cases achieved by the securing of a para-occlusal attachment. The previously described systems have a considerable weight and, on account of long leverages, accordingly cause high interference forces during the movement measurement. Some of the previously described systems also sit on soft tissue of the head so that measurements may therefore be distorted.

DE 10 2012 104 373 A1 describes a method for capturing patient-individual movements of the mandible in relation to the maxilla in a plurality of degrees of freedom. DE 10 2012 104 373 A1 thereby describes methods for recording 3D surface data of the maxilla and of the mandible, which surface data are used, for example, for the triangulation of surface or for the non-uniform rational B-spline (NURBS) method. Volume data can accordingly be recorded by CT, MRI or DVT, while 2D data are obtained from photographs, video recordings or textures.

DE 10 2007 058 883 A1 describes an intraoral retainer for a camera or a lens for image transmission.

### SUMMARY

An aspect of the present invention is to make available a method which can be carried out simply, and a corresponding system which, while affording a high degree of comfort for the patient, allows intraoral measurements of jaw movements to be carried out with great precision in up to six degrees of freedom.

In an embodiment, the present invention provides a method for capturing patient-individual movements of a mandible in relation to a maxilla in a plurality of degrees of freedom. The method includes providing an optical sensor system comprising an image in an oral cavity of a patient, providing a defined object in the image of the optical sensor system in the oral cavity of the patient, bringing the optical sensor system into a fixed connection to the mandible or to the maxilla, capturing the defined object with the optical sensor system, the defined object having a defined relationship to the other maxilla or mandible, recording a sequence of spatial points of the defined object with the optical sensor system during a movement of the mandible, and storing the sequence of spatial points as a multi-dimensional movement line in a movement data record.

### BRIEF DESCRIPTION OF THE DRAWINGS

The present invention is described in greater detail below on the basis of embodiments and of the drawings in which:

FIG. 1 shows a schematic diagram of a system according to the present invention which is held in the oral cavity of a patient where an individually shaped retainer is clamped between the teeth of the maxilla and bears against the palate;

FIG. 2 shows a schematic diagram of a system according to the present invention which is held in the oral cavity of a patient where three intraoral cameras are received in the upper retainer and are directed at specific objects that are provided in a lower retainer;

FIG. 3 shows a schematic diagram of a system according to the present invention which is held in the oral cavity of a patient, which is optimized for the greatest possible number of degrees of freedom, where a retainer is fitted between the teeth of the maxilla;

FIG. 4 shows a schematic diagram of a system according to the present invention which is held in the oral cavity of a patient, which is optimized to provide the greatest possible clearance for the tongue of the patient and to provide corresponding wearing comfort, where a splint is individually adapted to the teeth of the maxilla and carries a retainer;

FIG. 5 shows a schematic diagram of a system according to the present invention which is held in the oral cavity of a patient which corresponds to a combination of the embodiments of FIGS. 1 and 3, where a retainer is fitted between the teeth of the maxilla and has a downwardly extending web in which three horizontally oriented intraoral cameras are integrated;

FIG. 6 shows a schematic diagram of a system according to the present invention which is held in the oral cavity of a patient where a retainer, which is fitted between the teeth of the maxilla, extends in an arc under the palate and thus leaves space for the tongue;

FIG. 7 shows a plaster model of an opened jaw where a re-usable camera module is held in a universal receptacle on a retainer specially formed in the mandible;

FIG. 8 shows a variant in which a therapy splint is placed onto the teeth of the mandible so that cameras incorporated therein are directed vertically upward to capture the movement of the opposing teeth in the maxilla;

FIG. 9 shows a schematic diagram of a system according to the present invention which is held in the oral cavity of a patient which is similar to that of FIG. 4, but which permits a measurement of the biting force via a flexible test body inserted into the recess, which test body is made of rubber and is acted on by the head of the stamp;

FIG. 10 shows a schematic diagram of a system according to the present invention which is held in the oral cavity of a patient in which two-dimensional photosensitive diodes are used which are able to detect the center of gravity of a point of light on an active surface;

FIG. 11 shows a schematic diagram of a system according to the present invention which is held in the oral cavity of a patient with two-dimensional photosensitive diodes in which the emitters of the opposing jaw have a lens; and

FIG. 12 shows a schematic diagram of a system according to the present invention which is held in the oral cavity of a patient with two-dimensional photosensitive diodes where diodes and emitters with a lens are located on the same jaw side.

### DETAILED DESCRIPTION

The core aspect of the present invention is that, with at least one optical sensor system which is held in a defined position in the oral cavity and which can comprise an intraoral camera or a photosensitive device such as a diode array or a PSD (position-sensitive device), an object which is likewise located in a defined position in the oral cavity, and which is formed by a light source in the case of the photosensitive device, is captured during the jaw movement or chewing movement, and the movement data are recorded in multi-dimensional space. The optical sensor system is anchored fixedly in the reference system either of the maxilla or of the mandible, while the object to be captured is located in the reference system of the correspondingly opposing mandible or maxilla. To simplify matters, it is assumed below that the optical sensor system is held fixedly at a defined position of the maxilla. In the case of the intraoral camera, the object to be captured can be part of the anatomy of the oral cavity, for example, a tooth. A separate marking is, however, advantageously fixed as an object in the mouth. In the case of the photosensitive device, at least one light source, in particular with a strongly focused beam, is held in a defined position in the oral cavity of the patient.

According to the present invention, a sequence of spatial points of the object is recorded by the optical sensor system during the movement of the mandible and is stored as a correspondingly multi-dimensional movement line in a movement data record. It is assumed in the case of the intraoral camera that sufficient light is available in the oral cavity for capturing the images. The light can be supplied from outside or can advantageously be generated inside the oral cavity. Since the movements are captured by the optical sensor system, it is in particular possible for lateral movements to be detected with suitably high resolution. Forward and rearward movements can also be detected by the one or more optical sensor systems.

After suitable registration and merging, the movement data can then be used to animate a 3D computer graphics model of the jaw region of the patient to a virtual movement. The real patient-individual movements of the mandible in the 3D volume can thereby be presented on a screen in a manner faithful to the anatomy. The movement tracks of the temporomandibular joint can be visualized for each point in the 3D volume.

A particular advantage of the intraoral optical sensor system according to the present invention is of course that no rigid retainers must be extracted from the oral cavity and that the patient retains his/her freedom of movement during the mastication movements. Sufficient space also remains for the tongue depending on the embodiment, so that the patient does not suffer any discomfort during image capture.

For recording the multi-dimensional movement line, a single optical sensor system suffices if its images are evaluated photogrammetrically. The aim of such a photogrammetric evaluation is the restoration of images to the spatial position relative to each other in which they were located at the time of the image capture. This restoration is effected according to the laws of central projection while observing the condition of co-planarity.

In order to improve resolution, it may be advantageous, however, if the optical sensor system located in the oral cavity is equipped with a plurality of intraoral cameras or photosensitive devices which, in the simplest case, capture the same object from various perspectives during the movement. It is also possible, however, for each intraoral camera or photosensitive device to be assigned its own object. The procedure according to the present invention in this case is in principle also independent of whether an intraoral camera or a photosensitive device is looking “up” toward the maxilla and the other intraoral camera or the other photosensitive device is looking “down”. The respectively captured movement data can then subsequently be superposed by computation on the basis of known relationships.

It is advantageous to use miniaturized intraoral cameras or photosensitive devices which transmit their images via a radio link, for example, via Bluetooth or WLAN, to a receiver located outside the oral cavity, where they are processed. Miniaturized cameras/photosensitive devices which have a suitable transmission functionality and which can be used for these purposes are available at reasonable prices on the market. Cameras are advantageously used which make use of a dedicated and likewise miniaturized light source. When using autonomous systems of this kind, no power line needs to be routed out of the oral cavity so that movements can be captured without difficulty all the way to the terminal occlusion. Further advantages of autonomous intraoral systems of this kind are that they are relatively light and therefore do not distort the mastication movement on account of excessive weight. Laugh lines can also be captured without disturbance.

The particular advantage of the procedure according to the present invention is that movements can be captured in up to six degrees of freedom, three in translation, and three in rotation, and also as far as the occlusion. This is permitted by knowledge of the spatial relationship between the optical sensor systems and the objects.

The system is advantageously connected directly and rigidly to the teeth of the maxilla and/or the mandible so that image distortion caused by connections to the soft tissue is excluded. In an embodiment, this can, for example, be achieved via individually shaped retainer bows which are formed in the mandible and the maxilla of the patient. The use of the retainer bows advantageously entails the following workflow:

Firstly, an intraoral camera captures images of both rows of teeth. The result of this image capture is highly precise 3D surface data of the teeth. If appropriate, the buccal occlusion is also captured in order to subsequently be able to optimize the placement of the sensors. Planning must generally take into account that the bows do not interfere with antagonists.

On the basis of the data originating from the images, retainer bows are printed which can be introduced lingually into the maxilla and the mandible of the patient. Through precise knowledge of the surface data of the teeth, the retainer bows can be fitted precisely into the patient-individual jaws. The maxillary bow is connected to a measurement module, in particular having a plurality of intraoral cameras and light sources. The object to be captured is a pointer which is secured on the mandibular bow and with which the movements can be measured. The finished retainer bows are introduced into the patient's mouth. In some circumstances, further images are indicated if the retainer bows are fixed to the teeth, in order to detect their final position. This is not necessary if the terminal occlusion is captured as reference position. The measurement module and the pointer as object are secured on the fixed retainer bows. After the start of the measurement, the captured data are sent directly by short-range radio and in real time to the external receiver and registered in the computer. It is advantageous here for the intraoral cameras to be held at different angles in order to be able to capture all degrees of freedom with high resolution.

A particular advantage of the retainer bows is that they support a modular configuration of the system. The fitted measurement module can in particular be re-used for other measurements. The retainer bow of the next patient is therefore provided with a suitably standardized receptacle. The retainer bows also afford good retention since they can be pressed against the teeth from the inside with an interference fit.

The retainer bows can be provided with radiopaque markers in order to improve the registration of the system with the three-dimensional data records created in advance. With an additional printed part, the procedure according to the present invention also makes it possible to print an articulation register that is clipped onto the retainer bows for each recorded jaw position or for each jaw position set artificially in the simulation.

The autonomous and miniaturized system according to the present invention can also be supported in a digital volume tomograph (DVT) and/or face scanner device without an obstructive device being seen outside the mouth. The soft-tissue movements visible in the face scanner can be captured with the tooth movements free of interference. The lips are closed. A soft-tissue model can thus also be created without external interferences.

Previously used therapy splints can also be employed to mount the system according to the present invention. The original splints are thereby complemented by sensor receptacles, or the sensors are affixed to the splint. The position of the sensors in relation to the splint can be determined by a scan. The sensors can also be mounted on an orthodontic splint so that the function/force measurement can also be performed with a brace.

In an embodiment, the optical intraoral cameras or photosensitive devices can, for example, be supported by acceleration sensors or gyroscopes which are assigned to the sensor module. It is thereby advantageous to fix the sensor module in the mandible since the mandible moves more than the maxilla. If acceleration sensors are provided in both jaws, it is possible to eliminate movements of the patient's head from the calculation.

If a bite elevation is planned, maxillary and mandibular splints can also be prepared which simulate this bite elevation. The sensors for the movement measurement are mounted on the bite elevation splints so that it is possible to test the function of the patient with the planned bite elevation.

In an embodiment of the present invention, the individually shaped retainer bows can, for example, be equipped with a clamping device, for example, a spring suspension, which additionally favors a rigid connection to the teeth even without adhesive connections to the teeth having to be produced.

In an embodiment of the present invention, the individually shaped retainer bows can, for example, have a device for measuring deformations of the bows. Very slight movements of the individual teeth can thereby be detected and taken into account in the movement model. By taking account of the individual movements of the teeth, it is possible to provide a more exact prediction of the actual terminal occlusion. The contact points between the teeth of the terminal occlusion can therefore be predicted with greater accuracy. The individually shaped retainer bows can be constructed, for example, so that a means for changing position between the individual retainer members is placed in each case between two adjacent teeth.

The optical sensors are able to determine the center of gravity of an incident point of light with great precision if they are configured as photosensitive diodes, particularly arranged in an array, or as PSDs (position-sensitive devices/detectors). Such PSDs are optical position sensors (OPS) which are able to measure the one-dimensional or two-dimensional position of a point of light. PSDs work as analog sensors, which have an isotropic sensor surface and deliver continuous position information, or as discrete sensors, of which the surface is structured like a grid and which therefore deliver discrete position information.

In contrast to the intraoral camera, no digital image is evaluated in the case of the photosensitive device; the position of the point of light along two coordinate axes on the sensitive surface is instead determined on the basis of two analog voltage differences. Through the use of a plurality of planar sensors and through the use of a plurality of light-emitting sources in the opposing jaw, the position of the jaws in relation to each other can be determined in six degrees of freedom by a triangulation method. The advantages of photosensitive devices are the compact structure and also the very high position resolution of a few nanometers and the very high time resolution of a few nanoseconds. In contrast to a camera sensor, no complicated reworking of the signals is necessary. The disadvantage of the photosensitive device is that the center of gravity of all incident light is determined, which makes it necessary to focus the emitter light and to avoid scattered light. To be able to determine the position of a plurality of emitters on the surface of a single diode, the emitters must be controlled with a time lag so that only one emitter emits light at any one time, since otherwise the center of gravity of all visible emitters would be determined as position. At each time, therefore, only a single emitter transmits light. In an embodiment of the present invention, the emitters can, for example, transmit the light in a time pattern. It is clear via this time pattern from which emitter the light on the diode surface originates. A time pattern can, for example, involve each emitter transmitting with a fixed flash rate.

The present invention is described in more detail below under reference to the drawings. All of the drawings show a particular configuration of the system according to the present invention.

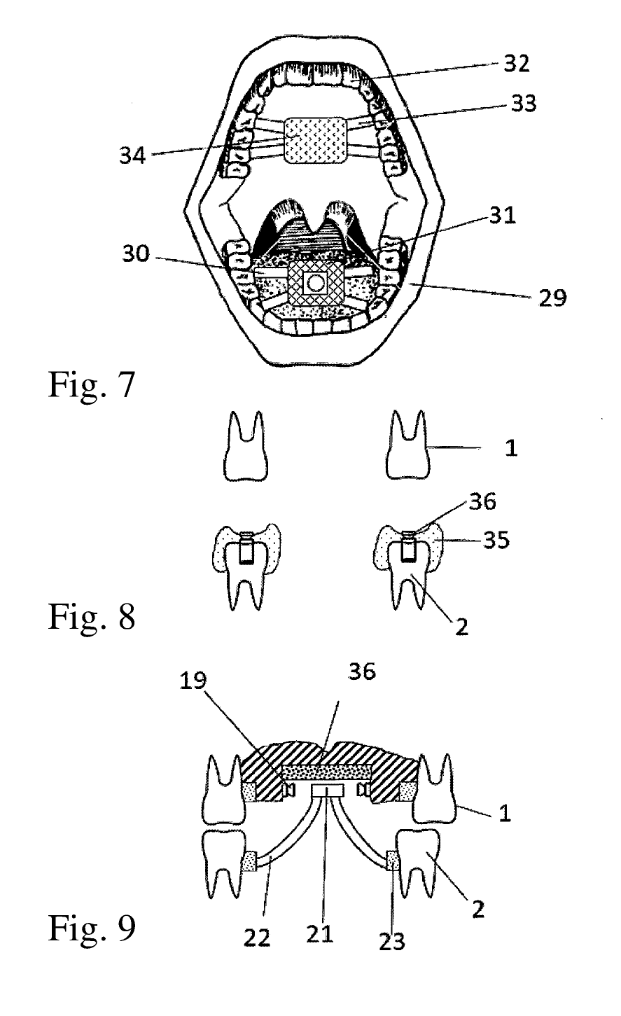

With the exception of FIG. 7, all of the drawings show schematic diagrams of systems according to the present invention which are held in the oral cavity of a patient. A view looking from the front into the mouth is shown in each case, the only anatomical features depicted being the teeth 1 of the maxilla and the teeth 2 of the mandible. The palate 3 is shown only indirectly.

FIG. 1 shows a variant of the system according to the present invention, in which an individually shaped retainer 4 is clamped between the teeth 1 of the maxilla and bears against the palate 3. A power source 5 and a Bluetooth transmitter 6 are integrated in the retainer 4. The Bluetooth transmitter 6 transmits to the exterior the signals that it receives from two intraoral cameras 7. The two intraoral cameras 7 are oriented obliquely downward at an angle and each have prominent structures of a mandibular tooth 2 in their sight. The intraoral cameras 7 are also each assigned a light source. Two optical sensors systems are thus provided in this case, each of them comprising an intraoral camera 7 and a tooth with defined structure located in the image of the intraoral camera 7. From the movements made by the teeth during chewing movements, two sequences of spatial points are recorded from which a multi-dimensional movement line can be established. In order to improve the resolution, it is also possible to spray defined patterns onto the teeth in order to better highlight the contour of the teeth.

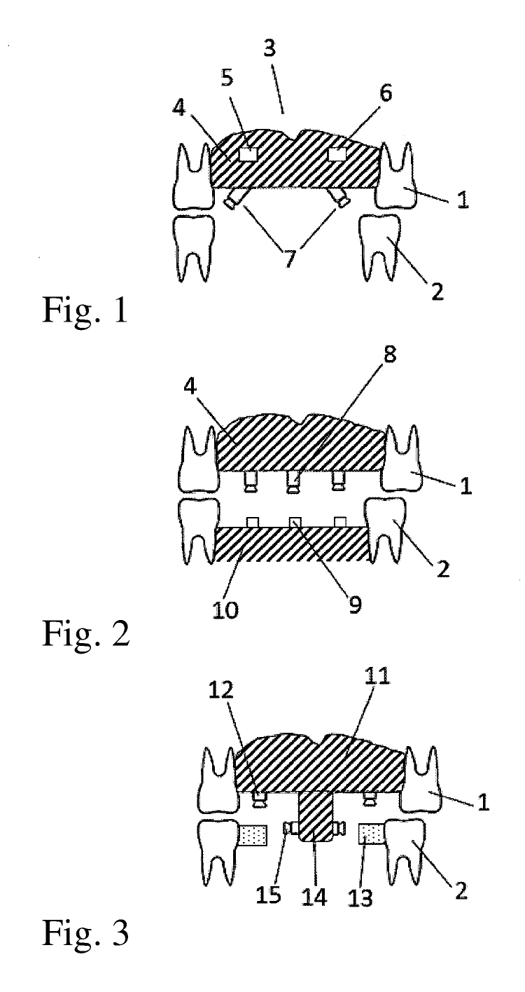

In the example according to FIG. 2, three intraoral cameras 8 are received in the upper retainer 4 and are directed at specific objects 9 (“pointers”/“markings”) that are provided in a lower retainer 10. The lower retainer 10 is likewise shaped individually to the teeth 2 of the mandible and accordingly clamped between the mandibular teeth 2. The objects 9 can be raised structures, as shown here, or they can be formed by a two-dimensional pattern, for example a QR code or barcode or some other image pattern.

FIG. 3 shows an embodiment which is optimized, in terms of image capture, for the greatest possible number of degrees of freedom, in particular also of the vertical movements. Once again, a retainer 11 is fitted between the teeth 1 of the maxilla. In the same way as in the above embodiment according to FIG. 2, two intraoral cameras 12 are oriented so that they point vertically downward. The object at which they are directed is a splint 13 which is prepared individually and clamped lingually between the mandibular teeth 2 of the patient. The surfaces of the splint 13 can be covered with an image pattern. However, the retainer 11 has a downwardly extending web 14, in which two intraoral cameras 15 are likewise integrated. The intraoral cameras 15 are each directed horizontally, to the left and right respectively, toward the side wall of the splint 13, which side wall can likewise be covered with an image pattern. This embodiment is particularly suitable for capturing vertical, forward, and rearward movements with high resolution, while the interoral cameras 12 are better able to capture movements to the front and rear and to the left and right.

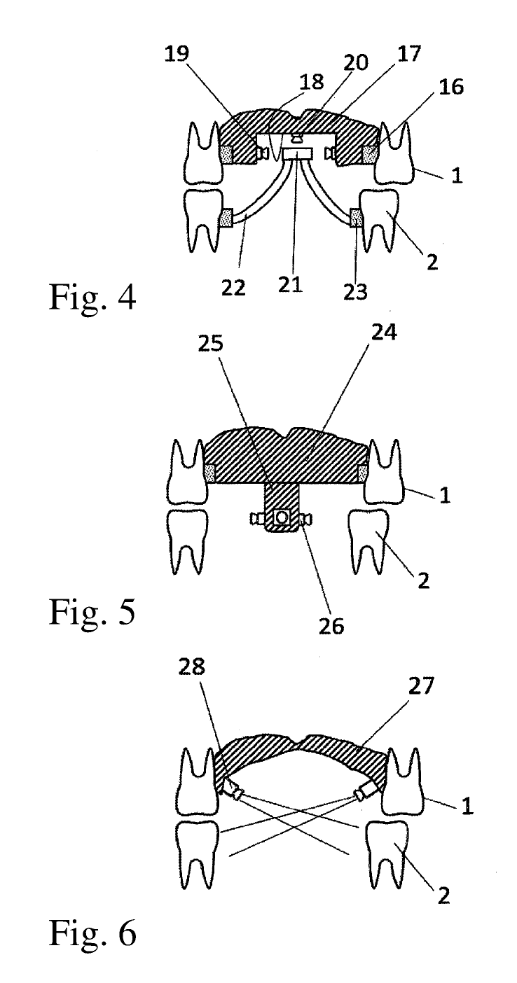

The embodiment according to FIG. 4 is optimized to give the greatest possible clearance for the tongue of the patient and to provide corresponding wearing comfort. A splint 16 is individually adapted to the teeth 1 of the maxilla and carries a retainer 17. A downwardly open recess 18 is formed in the retainer 17, the interior of which recess 18 is illuminated by two intraoral cameras 19 oriented horizontally to left and right. An intraoral camera 20 is oriented vertically downward. The head 21 of a stamp engages in the recess 18, which stamp is fixed via struts 22 to a splint 23 held on the mandibular teeth 2. Image patterns are again applied on the surface and on the sides of the head 21 and are recorded by the intraoral cameras 19 and 20 during movements of the jaw. The tongue of the patient lies comfortably between the struts 22 of the upwardly protruding stamp. All of the patient-individual parts can be printed.

The embodiment according to FIG. 5 corresponds to a combination of the embodiments from FIGS. 1 and 3. A retainer 24 is fitted between the teeth 1 of the maxilla and has a downwardly extending web 25 in which three horizontally oriented intraoral cameras 26 are integrated. The intraoral cameras 26 each have prominent structures of a mandibular tooth 2 in their sight. The movement of the teeth recorded as objects is thus observed from the side, so that vertical movement components can be better identified.

Similar to FIG. 5, the variant according to FIG. 6 has a retainer 27 which is fitted between the teeth 1 of the maxilla, which retainer 27 extends in an arc under the palate and thus leaves space for the tongue. At the lower edge of the arc, two intraoral cameras 28 are provided which are directed obliquely downward toward mutually opposite mandibular teeth 2. In contrast to the embodiment according to FIG. 4, no stamp extends downward from the maxilla.

FIG. 7 shows a plaster model of an opened jaw. A re-usable camera module 31 is held in a universal receptacle on a retainer 30 specially formed in the mandible 29. The cameras integrated in the camera module 31 are directed upward, where an image pattern 34 is printed on a retainer 33 formed in the maxilla 32. Movement lines can be recorded in six degrees of freedom with this embodiment.

FIG. 8 shows a variant in which a therapy splint 35 is placed onto the mandibular teeth 2. Cameras 36 incorporated therein are directed vertically upward and capture the movement of the opposing teeth 1 in the maxilla. The therapy splint 35 is thus in a form-fit engagement with the mandibular teeth 2 and, on the other side, forms a defined contact for the teeth 1 of the maxilla.

FIG. 9 shows a variant which is similar to that of FIG. 4 and accordingly uses the same reference signs, but which permits a measurement of the biting force via a flexible test body 36 inserted into the recess 18, which test body 36 is made of rubber and is acted on by the head 21 of the stamp. The interoral cameras 19 capture the compression of the test body 36 with known material properties.

The forces close to occlusion can thus be determined by the finite element method (FEM). The lateral cameras 19 remain free and can measure the vertical compression of the test body 36. Movements to the left and right can be measured by front cameras. The upper cameras are concealed by the test body 36. Since the position of the terminal occlusion is known, either the measurement indicator in the mandible can be adapted or the test body 36 can be pressed to the correct thickness.

With different thicknesses and Shore hardnesses of the rubber, it is possible for measurements to be carried out close to the occlusion with different dynamics. By virtue of the high spatial resolution of the cameras, the measurement system can resolve a very fine quantization of the forces through the FEM simulation.

The rubber can be replaced by a piezo force measurement module, which is likewise inserted into the recess at the palate. Contacts for the piezo force measurement element are present in the recess. The indicator in the mandible is configured so that it makes contact with the force measurement element close to occlusion. The mastication force can thus be measured in occlusion without interferences.

Rubber and the piezo sensor can also be combined. The support on the palate is good in the vertical direction so that high forces can also be measured.

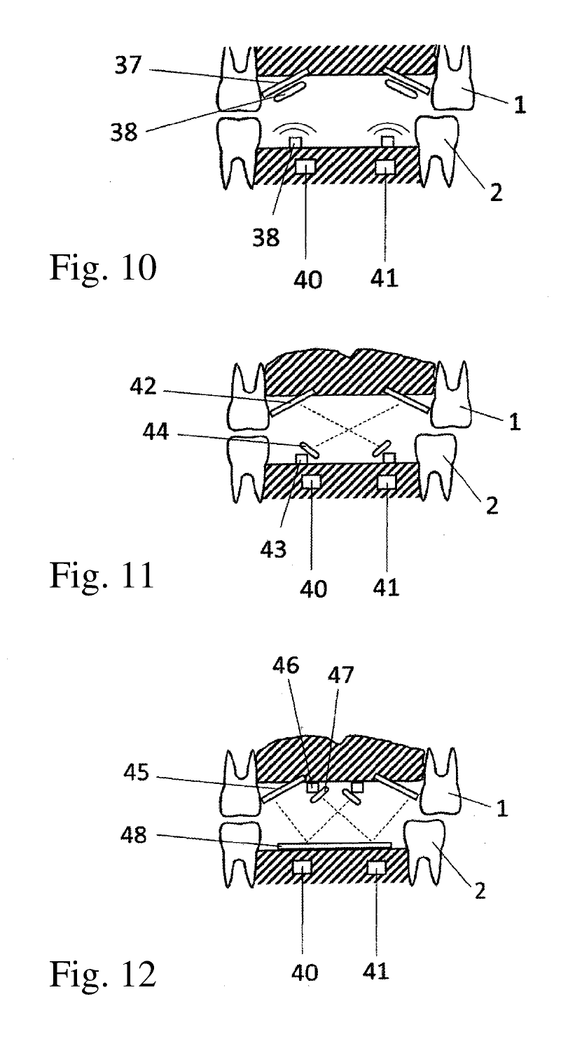

FIG. 10 shows an embodiment in which two-dimensional photosensitive diodes 37 are used which are able to detect the center of gravity of a point of light on an active surface. The surface diode has a lens 38 which focuses incident light from a plurality of actively illuminating emitters 39 in the opposing jaw. The opposing jaw contains an energy source 40 and control electronics 41 for the emitters 39. The emitters 39 are controlled via a time or frequency modulation in order to be able to establish from which emitter 39 a point of light on a diode 37 was sent.

FIG. 11 shows an embodiment with two-dimensional photosensitive diodes 42 in which it is not the diodes but the emitters 43 of the opposing jaw that have a lens 44. The lens 44 provides that the light of the emitter 43 is focused and that a focused image point is applied to the surface diodes and its center of gravity can be determined. Similarly to FIG. 10, the emitters 43 are controlled via a time or frequency modulation. Instead of using emitters with a focusing lens, it is also possible to use lasers that already emit focused light.

FIG. 12 shows an embodiment with two-dimensional photosensitive diodes 45, in which diodes 45 and emitters 46 with a lens 47 are located on the same jaw side. The light signals emitted by the emitters 46 are focused by the lens 47 and returned by reflectors 48 on the opposing jaw side so that they are projected as points of light on the surface diodes. The advantage of this embodiment is that the opposing jaw side can be configured as a passive module that requires neither an energy source nor control electronics for emitters. Instead of using emitters with focusing lenses, it is also possible to use lasers that already emit focused light. The reflectors 48 on the opposing jaw side can be configured both as mirror or prism-type reflectors and also as retroreflectors. Retroreflectors have the property of returning the incident light to the light source in the same direction or a similar direction.

The present invention is not limited to embodiments described herein; reference should be had to the appended claims.

What is claimed is:

1-15. (canceled)

16. A method for capturing patient-individual movements of a mandible in relation to a maxilla in a plurality of degrees of freedom, the method comprising:
providing an optical sensor system comprising an image in an oral cavity of a patient;
providing a defined object in the image of the optical sensor system in the oral cavity of the patient;
bringing the optical sensor system into a fixed connection to the mandible or to the maxilla;
capturing the defined object with the optical sensor system, the defined object having a defined relationship to the other maxilla or mandible;
recording a sequence of spatial points of the defined object with the optical sensor system during a movement of the mandible; and
storing the sequence of spatial points as a multi-dimensional movement line in a movement data record.

17. The method as recited in claim 16, wherein the optical sensor system is an intraoral camera.

18. The method as recited in claim 16, wherein,
the optical sensor system is a photosensitive device, and
the defined object is at least one light source comprising a focused beam.

19. The method as recited in claim 18, wherein the photosensitive device is a diode array or a position-sensitive device (PSD).

20. The method as recited in claim 16, further comprising:
a plurality of optical sensor systems in a defined relationship to one another; and
calculating a three-dimensional movement line located in the oral cavity from a superposition of the movement data record captured by the plurality of optical sensor systems.

21. The method as recited in claim 16, further comprising:
providing a 3D computer graphics model of a jaw region of the patient; and
applying the movement data record to the 3D computer graphics model of the jaw region of the patient so as to provide an animation of a corresponding virtual movement.

22. The method as recited in claim 16, further comprising:
sending the sequence of spatial points recorded by the optical sensor system via a radio connection to a receiver located outside of the patient.

23. The method recited in claim 16, further comprising:
fixing a compressible test body on a retainer to one of the jaws;
providing a stamp held on an opposing jaw to the compressible test body;
subjecting the compressible test body to a force via the stamp under an effect of a bite;
capturing a deformation of the compressible test body via intraoral cameras; and
determining a biting force from images of the interoral cameras.

24. The method as recited in claim 23, wherein the compressible test body is made of rubber.

25. The method as recited in claim 16, further comprising:
preparing at least one retainer which is configured as a retainer bow to retain the optical sensor system in at least one of the mandible and the maxilla of the patient based on three-dimensional surface data of the teeth.

26. A system for an intraoral capture of patient-individual movements of a mandible in relation to a maxilla in a plurality of degrees of freedom, the system comprising:
an optical sensor system configured to be releasably secured in an oral cavity of a patient, the optical sensor system comprising an image;
a defined object located in the image of the optical sensor system, the optical sensor system being configured to record a movement of the mandible as a sequence of spatial points of the defined object; and
a storage device configured to store the sequence of spatial points as a multi-dimensional movement line in a movement data record.

27. The system as recited in claim 26, wherein the patient-individual movements are chewing movements.

28. The system as recited in claim 26, wherein the optical sensor system comprises an intraoral camera.

29. The system as recited in claim 26, further comprising:
a receiving and evaluation unit arranged outside the oral cavity; and
a transmitting device configured to transmit at least one of the sequence of spatial points and the movement data record to the receiving and evaluating unit.

30. The system as recited in claim 26, wherein,
the optical sensor system comprise a photosensitive device, and
the defined object is provided as at least one punctiform light source arranged in the oral cavity of the patient.

31. The system as recited in claim 30, wherein the photosensitive device is a diode array or a position-sensitive device (PSD).

32. The system recited in claim 30, further comprising:
a camera module; and
at least one retainer which is individually configured to at least one of the mandible and the maxilla, the at least one retainer comprising a universal receptacle for the photosensitive device or for the camera module.

33. The system as recited in claim 32, wherein the at least one retainer further comprises a clamping device configured to connect the at least one retainer to teeth of the patient.

34. The system as recited in claim 33, wherein the clamping device is a spring suspension.

35. The system as recited in claim 32, further comprising:
a measuring device configured to measure at least one deformation of the at least one retainer.
